(19) United States
(12) Patent Application Publication
(10) Pub. No.: US 2012/0323257 A1
(43) Pub. Date: Dec. 20, 2012

(54) Soft-pedicare

(76) Inventor: BerNArd S. Sutton, Atlanta, GA (US)

(21) Appl. No.: 13/506,938

(22) Filed: May 25, 2012

Related U.S. Application Data
(60) Provisional application No. 61/519,948, filed on Jun. 2, 2011.

(51) Int. Cl. A61B 17/32

(52) U.S. Cl. 606/131

(57) ABSTRACT

A sponge rubber foot exfoliation apparatus to remove thick, rough, dry, peeling, flaky skin and calluses from the arches, tops, bottoms, sides, heels and toes of feet evenly gently painlessly, smoothly and softly. The first apparatus consisting of a sponge rubber handheld device will attach to a second apparatus. The second apparatus comprising flexible material with abrasives and sponge rubber material affixed with abrasives to exfoliate feet. A single rigid plastic perimeter ring, double rigid plastic plates and single rigid plastic plate of the second apparatus will attach to and secure the flexible and sponge rubber material with abrasives to the first apparatus.

### 1. CROSS REFERENCE TO RELATED APPLICATIONS

The instant non-provisional application claims priority from provisional application No. 61/519,948 filed Jun. 2, 2011, and entitled “Soft-Pedicare”

### 2. BACKGROUND OF THE INVENTION

A. Field of the Invention

The present invention relates to a foot exfoliation apparatus, and more particularly, the present invention relates to a foot exfoliation apparatus to exfoliate rough, thick, flaky, dead, dry skin and calluses from feet evenly, gently, painlessly, smoothly and softly.

B. Description of the Prior Art

The Soft-Pedicare apparatus is in the category of foot files, callus shavers and callus removers that cut into, grate, scrape and file away rough, thick, dry, flaky, peeling skin and calluses from feet. Callus removers have raised mini razor-like-blades and callus shavers use double-edge razor blades. The prior art causes a great deal of pain, discomfort, leave feet very sore and must be used properly to avoid very serious injury to feet and hands. The prior art have narrow, thin, flat, hard plastic handles and surfaces that do not conform to the contours, curves, bending joints, pressure points and shapes of fingers and palms when gripping, holding and using them. None of the prior art will conform to the contours, curves, bending joints, pressure points and shapes of arches, ankles, bottoms, sides and heels of feet and toes. None of the prior art will flex, gyrate or pivot with the natural gyrations and movements of fingers, hands and wrists. U.S. D596,802 S is an example of prior art that consist of a metal plate with raised mini razor-like-blades, plastic bubble skin receptacle, very thin mini-abrasive discs and other hard plastic parts seen in “Ped-Egg”, “For Her Smooth My Sole”, “Ped-Pro”, “Pedi-Expert” and “Flawless Pedicure” products available to consumers in retail outlets. Additional prior art are shown in U.S. Pat. Nos. 3,196,885, 6,740,052, Des. 419,724, U.S. D442,741 S, U.S. D486,607 S, U.S. D509,027 S, U.S. D530,859 S, U.S. D623,801 S, U.S. D631,199 S and U.S. D634,070 S.

### 3. SUMMARY OF THE INVENTION

Accordingly, an object of the present invention is to provide a foot exfoliation apparatus to exfoliate feet of rough, thick, flaky, dry, peeling skin and calluses that avoids the disadvantages of the prior art.

Briefly stated, another object of the present invention is to provide a foot exfoliation apparatus to remove rough, thick, flaky, dry, peeling skin and calluses from feet evenly, gently, painlessly, smoothly and softly. Soft-Pedicare has a first apparatus and a second apparatus. The first apparatus comprises a non-abrasive sponge rubber handheld device. The second apparatus comprises abrasive material affixed on sponge rubber material attached to a rigid plastic plate separable from the handheld portion and a second embodiment of the second apparatus is comprised of abrasives affixed on flexible material attached to a rigid plastic perimeter ring separable from the sponge rubber handheld apparatus, both embodiments comprising the second apparatus which are detachable abrasive devices to exfoliate feet.

Another object of the invention comprises a sponge rubber handheld apparatus that will conform to the contours, curves, bending joints, pressure points and shapes of fingers and hands, the sponge rubber material reducing fatigue and stress to fingers, hands and wrist.

Another object of the invention comprises a handheld sponge rubber apparatus that will flex, gyrate and pivot with the natural gyrations and movements of fingers, hands and wrists.

Another object of the invention comprises detachable abrasive sponge rubber material affixed with abrasives and detachable flexible material covers affixed with abrasives attachable to contoured sponge rubber material both embodiments conforming to the curves, contours, bending joints, pressure points and shapes of feet when pressed onto feet.

Another object of the invention comprises a foot exfoliation apparatus that will be easy and simple to use.

Another object of the invention comprises a foot exfoliation apparatus that is economical in costs to manufacture.

The novel features which are considered characteristic of the present invention is set forth in the appended claims. The invention itself, however, both as to its construction and its methods of operation, together with additional objects and advantages, will be best understood from the following description of the specific embodiments when read and understood in connection with the accompanying drawing.

Further objects of the invention will appear as the description proceeds. To the accomplishment of the above and related objects, the invention my be embodied in the forms illustrated in the accompanying drawings, attention being led to the fact, however, that the drawings are illustrative only, and that changes may be made in the specific construction illustrated and described within the scope of the appended claims.

### 4. BRIEF DESCRIPTION OF THE DRAWING

The figures of the drawing are briefly described as follows:

FIG. 1 is a diagrammatic side-view of a singular handheld apparatus having a half-oval bottom portion comprised of sponge rubber material of first apparatus of the present invention;

FIG. 2 is a diagrammatic side-view of an abrasive foot exfoliation apparatus that is attachable to FIG. 1, FIG. 2 comprising a top rigid plastic perimeter ring attached to flexible material with abrasives of first apparatus of the present invention;

FIG. 3 is a partial cross-sectional view of FIG. 2 taken along lines 4-4 that shows material and structural details of FIG. 2 of first apparatus of the present invention;

FIG. 4 is a top-down view of FIG. 2 taken along lines 2-2 that shows details of the rigid plastic perimeter ring and non-abrasive top-side of first apparatus of the present invention;

FIG. 5 is a back-rear view of FIG. 1 taken along line 3-3 that shows details of the sponge rubber material handheld device of first of apparatus of the present invention;

FIG. 6 is a second embodiment of apparatus 38 FIG. 1, FIG. 6 comprising a handheld portion, two rigid plastic plates with vertical extensions, indentations, sponge rubber material with abrasives of first apparatus of the present invention;

FIG. 7 is a third embodiment (cross-sectional) of the abrasive foot exfoliation pad comprising a rigid plastic bowl, top spiral extension, non-spiral extension, sponge rubber material with abrasives of first apparatus of the present invention;

FIG. 8 is a second embodiment of a rigid plastic device for the abrasive pad shown in FIG. 6, FIG. 8 comprising a spiral receptor with a wider radius for receiving a second spiral extension with a narrower radius shown in FIG. 7 or other spiral extension of first apparatus of the present invention;

FIG. 9 is a cross-sectional of the abrasive padded portion of FIG. 6 taken along line 5-5 that shows a rigid plastic plate and sponge rubber material with abrasives of first apparatus of the present invention;

FIG. 10 is a third embodiment of apparatus 38 FIG. 1, apparatus 240 FIG. 10 comprising a similar handheld device with elevated ridges, a single rigid plastic plate, individual sponge rubber pillows with abrasives, the pillows compressing upward and extending downward adjusting to feet of first apparatus of the present invention;

FIG. 11 is a side-view of a fourth embodiment of a foot exfoliation device apparatus 90 FIG. 6, FIG. 11 comprising double circular sponge rubber pillows with abrasives of first apparatus of the present invention;

FIG. 12 is a bottom-view of FIG. 11 of first apparatus of the present invention.

### 5. LIST OF REFERENCE NUMERALS UTILIZED IN THE DRAWING

### A. General

apparatus 38 FIG. 1 for exfoliating feet comprising sponge rubber material 69, center-palm oval dome 14, wide surface 58A to fit into palm of a wide hand, oval dome 14 and narrow hourglass waist 8A attached at 1A, roundly indented finger loop 19A and 22A will compress around and secure fingers, narrow hourglass waist feature 8A to allow handheld sections 22A, 8A, 19A, 58A and 14 comprising the narrow hourglass waist 8A and oval dome 14 to flex, gyrate and pivot with the natural gyrations and movements of fingers, hands and wrists, lateral finger groove 26A for extended thumb and four fingers (opposite sides) in a clutching and gripping position, perimeter line 30 extends continuously around perimeter of the device, perimeter ridge 23 extends continuously around perimeter of the device, tongue slots 32 and 34, half-oval base 18, bottom surface 16.

### B. First Embodiment of First Apparatus.

first apparatus 38, non-disposable device, center-palm circular dome 14, first apparatus 38, wider surface 58A to fit into palm of a wide hand, rounded indented finger loops 19A and 22A will compress around and secure fingers, narrow hourglass waist feature 8A, bottom of circular dome 14, top of circular narrow hourglass waist 8A are connected at perimeter line 1A lateral finger groove 26A for extended thumb and fingers, perimeter line 30 extends continuously around perimeter of the device, perimeter ridge 23 extends continuously around perimeter of the device, tongue slots 32 and 34 to receive tongue tabs, half-oval base 18, outer surface 16 of half-oval base.

### B. Figure Two

second apparatus 48, disposable device, rigid plastic perimeter ring 42 extends continuously around the perimeter of rigid plastic perimeter ring 42, attached 41,

to flexible material 47, abrasives 44, finger tab 36 to attach and remove apparatus 48, from apparatus 38 when finger tab 36 is pressed downward to release apparatus 48 from half-oval base 18 of apparatus 38.

### C. Figure Three

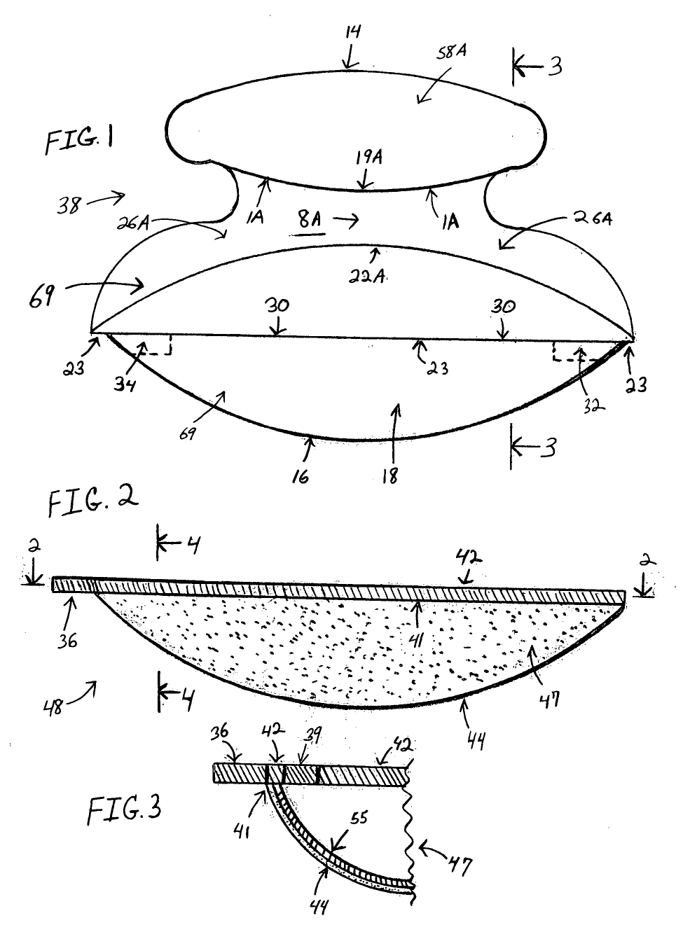

FIG. 3 partial cross-sectional view of apparatus 48 FIG. 2 taken along lines 4-4 showing rigid plastic perimeter ring 42, finger tab 36,

tongue tab 39,

tongue slot (34 FIG. 1),

attached 41,

non-abrasive flexible material 55, abrasives 44.

### D. Figure Four

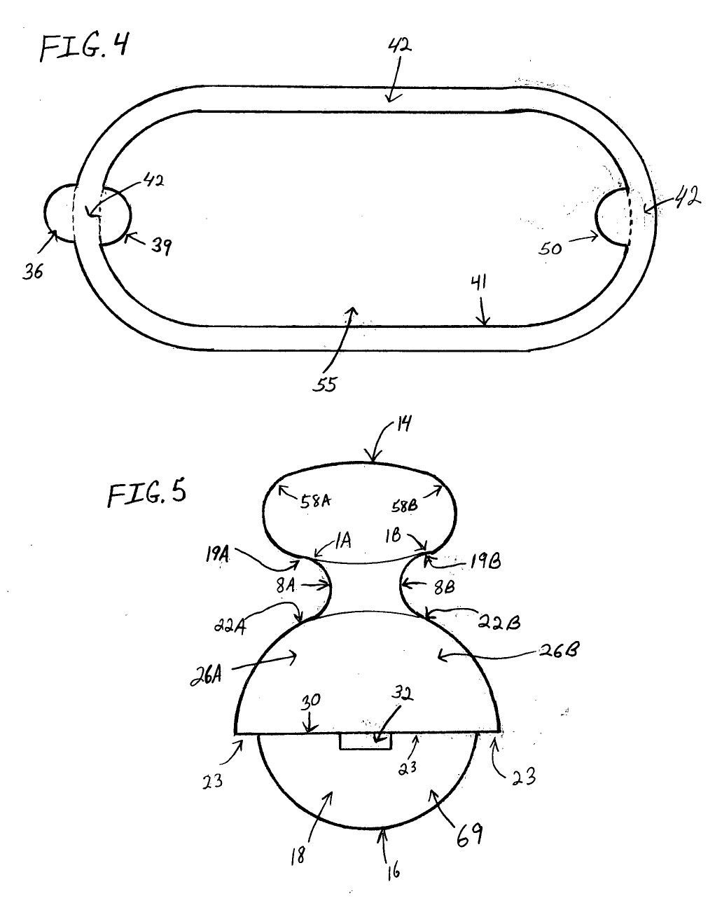

FIG. 4 top-down view of FIG. 2 taken along lines 2-2, rigid perimeter ring 42, attached 41, to topside of non-abrasive flexible material 55, finger tab 36, tongue tabs 39 and 50.

### E. Figure Five

FIG. 5 back-rear view of FIG. 1 taken along lines 3-3 comprising a center-palm oval dome 14,

wider surfaces 58A and 58B to fit into the palm of a wide hand, roundly indented finger loops 19A and 22A, and 19B and 22B will compress around and secure thumbs and four fingers (opposite sides), bottom of oval dome 14 and top perimeter edge of narrow hourglass glass waist 8A and 8B are attached at extending line 1A and 1B, narrow hour-glass waist features 8A and 8B will permit sections 22A, 8A, 19A, 58A, 14, 58B, 19B, 8B and 22B comprising the oval dome 14 and narrow hourglass waist 8A and 8B to flex, gyrate and pivot, lateral finger grooves 26A and 26B for extended thumb and fingers, half-oval base 18, outer surface edge 16, tongue slot 32 to receive tongue tab 50 (FIG. 4) to secure rigid plastic perimeter ring 42, below perimeter ring 30, against perimeter ridge 23.

### F. Second Embodiment of First Apparatus

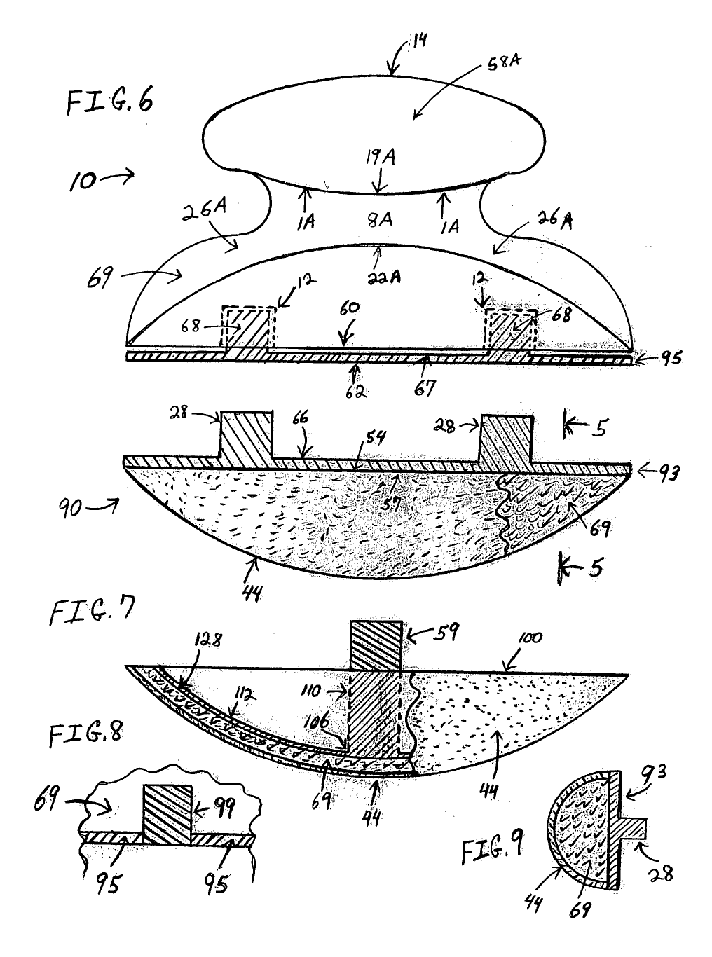

FIG. 6 apparatus 10 comprises non-abrasive sponge rubber 69 material, attached to rigid plastic plate 95, apparatus 90 comprises a half-oval portion of sponge rubber 69 material, affixed with abrasives 44, attached to rigid plastic plate 93, rigid plastic plate 95 has a top flat surface 67, bottom flat surface 62, hollow vertical extensions 68, will adhesively insert into indentations 12, bottom flat surface 60, of sponge rubber 69 material, will adhesively attach to the top flat surface 67, of rigid plastic plate 95, rigid plastic plate 93 has a top flat surface 66, bottom flat surface 54, rigid plastic extensions 28, atop flat surface 66, will detachably attach to hollow extensions 68, of rigid plastic plate 95, bottom 54, of rigid plastic plate 93, will adhesively attach to top 57, of sponge rubber material 69, covered with abrasives 44, bottom flat surface 62, of rigid plastic plate 95, and top flat surface 66, of rigid plastic plate 93 will lay together when rigid solid plastic extensions 28, on rigid plastic plate 93, are detachably inserted into rigid hollow plastic indentations 68, atop rigid plastic plate 95.

### F. Figure Seven

FIG. 7 third embodiment of apparatus 48 FIG. 2 and apparatus 90 FIG. 6 comprising

flexible sponge rubber, abrasive and rigid plastic materials, FIG. 7 comprises a rigid plastic bowl device with a top flat surface 100, spiral extension 59, bottom non-spiral extension 110,

connected 106, to top upwardly curved surface 112, of rigid bowl device, bottom upwardly curved 128, surface of rigid bowl device,

attached to sponge rubber material 69,

with abrasives 44

### G. Figure Eight

FIG. 8 second embodiment of FIG. 6, top rigid plastic plate 95,

FIG. 8 comprises a vertical spiral receptor 99 attached to rigid plastic plate 95, to receive a matching spiral vertical extension 59, in FIG. 7 will detachably spiral together of first apparatus.

### H. Figure Nine

FIG. 9 cross-sectional of apparatus 90 FIG. 6 taken along lines 5-5 shows a rigid plastic plate 93, rigid plastic extension 28 (side-view one shown),

sponge rubber material 69,

abrasives 44, of first apparatus.

### I. Third Embodiment of the First Apparatus

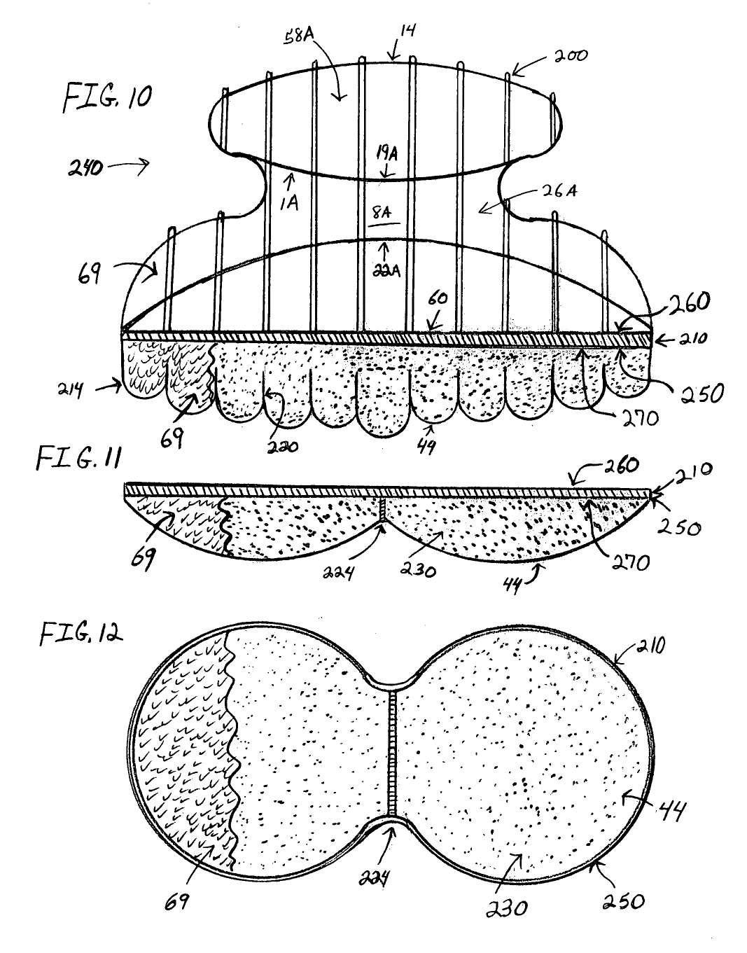

Apparatus 240 FIG. 10, single disposable device, third embodiment of first apparatus 38 FIG. 1, apparatus 240 comprises a handheld portion similar to handheld portions in FIG. 1 and FIG. 6, apparatus 240 FIG. 10 has elevated ridges 200 to prevent finger and hand slippage when apparatus 240 is held and moved in forward, backward, left, right and circular movements, handheld portion with elevated ridges adhesively attached to top flat surface 260 of rigid plastic plate 210, individual pillows 214 comprises sponge rubber material 69, abrasives 44, separated 220, attached to bottom 250 of rigid plastic plate 210.

### J. Figure Eleven

FIG. 11 side-view of a fifth embodiment of sponge rubber 69, and abrasives 44, sections of FIG. 1 and FIG. 2, FIG. 6, FIG. 7 and FIG. 10, FIG. 11 comprises double abrasive circular pillows 230, sponge rubber material 69, abrasives 44, attached to the rigid plastic bottom surface 250, of rigid material 210, separated 224, rigid plastic plate having a top flat surface 260.

### K. Figure Twelve

Is a bottom view of FIG. 11, double abrasive circular pillows 230, sponge rubber material 69, with abrasives 44, rigid material 210, separated at 224.

### 6. DETAILED DESCRIPTION OF THE PREFERRED EMBODIMENT

### A. General

Referring now to the figures, in which like numerals indicate like parts, and particularly to FIG. 1, is a diagrammatic side-view of the foot exfoliation apparatus to exfoliate thick, rough, flaky, dry, peeling skin and calluses from feet. (feet not shown). The first apparatus, a handheld device comprised of non-abrasive sponge rubber material and a second apparatus comprised of a rigid plastic perimeter ring with flexible material with abrasives will detachably attach to the first apparatus. The rigid plastic perimeter ring of the second apparatus is permanently attached to flexible material with abrasives on the outer surface and non-abrasive material on the inner surface when moved over feet will exfoliate thick, rough, flaky, dry peeling skin and calluses from feet evenly, gently, painlessly, smoothly and softly.

### B. First Embodiment of the First Apparatus

The configuration of the first embodiment of the first apparatus 38 can best be seen in FIG. 1 of a first embodiment of the first apparatus of the foot exfoliation device.

Apparatus 38 comprises a center-palm circular dome 14 to fit into the center palm of a hand, wider surface corner 58A to fit outer edges of a wide hand, narrow hourglass waist section 8A, for gripping and holding the circular dome to permit first apparatus 38 to flex, gyrate and pivot with the natural gyrations and movements of fingers, hands and wrists, bottom of circular dome 14 and narrow hourglass waist 8A are connected at line 1A.

Rounded indented finger loops 19A and 22A will compress firmly around and secure fingers when narrow waist section 8A is squeezed inward, perimeter line 30 extends continuously around the perimeter of apparatus 38, perimeter ridge 23 extends continuously around the perimeter of apparatus 38, below perimeter line 30 and perimeter ridge 23 are tongue slots 32 and 34, half-oval base 18, outer surface edge 16.

FIG. 2, comprises a rigid plastic perimeter ring 42 that extends continuously around the perimeter of the perimeter ring, attached 41, to flexible material 47, affixed with abrasives 44, rigid plastic perimeter ring 42 has a finger tab that will be held between fingers to attach and remove rigid plastic perimeter ring 42, from apparatus 38, below perimeter line 30 against perimeter ridge 23, single rigid plastic perimeter ring 42 has the width, radius and diameter of perimeter ridge 23, identically shaped half-oval flexible material 47, with abrasives 44, attached to perimeter ring 42 will attach to perimeter ridge 23 under perimeter line 30 matching the identically shaped half-oval base 18, sponge rubber 69 material of apparatus 38.

FIG. 3 is a partial cross-sectional of FIG. 2 taken along lines 4-4 that shows finger tab 36, rigid plastic perimeter ring 42, tongue tab 39, that will insert into tongue slot 34 (apparatus 38), non-abrasive surface of flexible material 55, bottom surface with abrasives 44, will attach 41, to rigid plastic perimeter ring 42.

FIG. 4 is a top-down view of FIG. 2 taken along lines 2-2 that shows finger tab 36 to attach or remove rigid plastic perimeter ring 42, tongue tabs 39 and 50 will insert into tongue slots 32 and 34 to detachably attach apparatus 48 on apparatus 38 below perimeter ring 30 against perimeter ridge 23, rigid plastic perimeter ring 42 has the width, radius and diameter of perimeter ridge 23 fitting around and over the half-oval base, FIG. 1.

FIG. 5 is a back-rear view of FIG. 1 taken along lines 3-3 that shows sponge rubber 69 material, center-palm oval dome 14, wider surface corners 58A and 58B to fit into palm of a wide hand, roundly indented finger loops 19A and 22A, and 19B and 22B will compress around and secure the thumbs and four fingers (opposite sides held), narrow hour-glass waist feature 8A and 8B attached to circular dome 14 at 1A and 1B will permit handheld sections 22A, 8A, 19A, 58A, 14, 58B, 19B, 8B and 22B comprising oval dome 14 and narrow hourglass waist feature 8A and 8B to flex, gyrate and pivot, lateral finger grooves 26A and 26B for extended thumb and fingers half-oval base 18, outer surface edge 16, tongue slot 32 will receive tongue tab 50 (FIG. 4) to secure rigid plastic perimeter ring 42 below perimeter ring 30 against perimeter ridge 23.

### C. Second Embodiment of the First Apparatus

FIG. 1 and FIG. 2, and FIG. 6 are similar (FIG. 5) except in the application and design of sponge rubber, abrasive and rigid plastic material parts.

Apparatus 10 FIG. 6 comprises a center-palm circular dome 14 to fit into the center palm of a hand, wider surface corners 58A and 58B to fit outer edges of a wide hand, narrow hourglass waist sections 8A and 8B for gripping and holding the device and to allow the handheld portion of apparatus 10 to flex, gyrate and pivot with the natural gyrations and movements of fingers, hands and wrists, rounded indented finger loops 19A and 22A, and 19B and 22B will compress firmly around and secure fingers when 8A and 8B are squeezed inward, lateral finger grooves 26A and 26B for extended thumb and fingers, vertical indentations 12, base 60, sponge rubber 69 material, rigid solid plastic plate 95, with rigid plastic hollow vertical extensions 68, will insert into sponge rubber indentations 12, adhesively attaching to the bottom 60, of the sponge rubber 69 materials portion and top flat rigid plastic surface 67, forming a single non-disposable handheld device comprising a sponge rubber handheld portion with a rigid plastic bottom portion, apparatus 90 FIG. 6 comprises a lower flat rigid plastic plate 93 with a top surface 66, vertical rigid plastic extensions 28, bottom flat surface 54 adhesively attached to top flat surface 57, sponge rubber material 69, with abrasives 44, forming a disposable exchangeable device, rigid solid plate 93 of apparatus 90 has rigid plastic solid extensions 28 on the top flat surface 66 to detachably insert into rigid plastic hollow extensions 68 when apparatus 90 is detachably attached to apparatus 10, rigid plastic plates 93 and 95 having the width, radius and diameter of sponge rubber 69 material flat surfaces 57 and 60.

FIG. 7 is a second embodiment of FIG. 1 and FIG. 2, and FIG. 6 for abrasive pads comprising flexible sponge rubber, abrasives and rigid plastic materials, FIG. 7 comprises a rigid plastic bowl shaped device with a flat top surface 100, rigid spiral extension 59, non-spiral extension 110, attached 106 to the top upwardly curved surface of bowl 112, rigid plastic bowl having a bottom upwardly curved surface 128, adhesively attached to sponge rubber material 69 with abrasives 44.

FIG. 8 is a second embodiment of apparatus 90 FIG. 6, rigid plastic plate 95, FIG. 8 comprising a single spiral extension 99 attached to a flat rigid plate 95 a spiral extension having a narrower radius 59, FIG. 7, which will spirally insert into spiral receptor 99 having a wider radius attached to rigid plate 95.

FIG. 9 is a cross-sectional of apparatus 90 FIG. 6 taken along lines 5-5 that shows the flat rigid plastic plate 93, rigid plastic extensions 28, sponge material 69 affixed with abrasives 44.

### D. Third Embodiment of the First Apparatus

FIG. 1 and FIG. 2, FIG. 6 and FIG. 10 are similar except in the application and design of the sponge rubber, abrasive and rigid plastic parts.

FIG. 10 apparatus 240 comprises a center-palm oval dome 14 to fit into the center palm of a hand, wider surface corners 58A and 58B to fit outer edges of a wide hand, narrow hourglass waist sections 8A and 8B for gripping and holding the apparatus and to allow the handheld portion of apparatus 240 to flex, gyrate and pivot with the natural gyrations and movements of fingers, hands and wrists, rounded indented finger loops 19A and 22A, and 19B and 22B will compress around and secure fingers when 8A and 8B are squeezed inward, lateral finger grooves 26A and 26B for extended thumb and fingers, apparatus 240 has elevated ridges 200 to prevent finger and hand slippage when apparatus 240 is held and moved in forward, backward, left, right and circular movements, apparatus 240 below line 60 has a single rigid plastic plate 210 with a top flat surface 260, flat bottom surface 250, individual sponge rubber 69 material pillows or rolls 214, abrasives 44, equally spaced 220 that will compress upward and extend downward adjusting and conforming to the curves, contours, bending joints, pressure points and shapes of feet when they are pressed onto feet, rigid plastic plate 210 having the width, radius and diameter of the flat sponge rubber 69 material surfaces 60 and 270, individual abrasive pillows 214 of apparatus 240 is a different embodiment of the flexible material, sponge rubber and abrasive portions of FIGS. 1, 2, 6 and 7.

### E. Fourth Embodiment of the First Apparatus

FIG. 11 comprises double circular abrasive pillows 230, sponge rubber material 69, abrasives 44, separated 224, attached to rigid material 210

FIG. 12 is a bottom view of FIG. 11 showing double circular pillows 230, sponge rubber material 69, abrasives 44 separated at 224.

It will be understood that each of the elements described above, or two or more together, may also find a useful application in other types of construction differing from the types described above.

While the invention has been illustrated and described as embodied in a disposable foot exfoliation apparatus for exfoliating feet, however, it is not limited to the details shown, since it be understood that various omissions, modifications, substitutions and changes in the forms and details of the device illustrated and its operations can be made by those skilled in the art without departing in any way from the spirit of the present invention.

The invention claimed is:

1. A foot exfoliation apparatus to remove rough, thick, flaky, dead, dry skin and calluses, the apparatus comprising:
a) a first device held by a clutched hand and gripping fingers;
b) a second device using various design and construction methods will attach to the second device to exfoliate rough, thick, flaky, dead, dry skin and calluses from feet.

2. The apparatus of claim 1, the first handheld device has an oval dome shape, the bottom portion not forming a complete circle, the extension of the incomplete oval extending downwardly.

3. The apparatus of claim 1, the first device includes a flexible narrow hourglass waist with circular vertical walls that extend upwardly around the perimeter of the flexible narrow hourglass waist, its top perimeter edge attaching to the bottom downward extension of the circular dome.

4. The apparatus of claim 1, the oval dome is attached to the top portion of a flexible narrow hourglass waist that extends around the perimeter and will flex, gyrate and pivot.

5. The apparatus of claim 1, the oval dome hand device has contoured finger grooves that extends outward laterally from the flexible narrow hourglass waist.

6. The apparatus of claim 1, the oval dome has contoured finger loops that will compress around and secure thumbs and fingers when the oval dome is clutched and flexible narrow hourglass waist is squeezed inwardly.

7. The apparatus of claim 1, the device has a perimeter line extends continuously around the perimeter of the perimeter line.

8. The apparatus of claim 1, the device has a perimeter ridge extends continuously around the perimeter of the perimeter ridge.

9. The apparatus of claim 1, the device has a half-oval base portion that projects downwardly from the continuous perimeter line and continuous perimeter ridge.

10. The apparatus of claim 1, the half-oval base has tongue slots that extends inwardly from the perimeter line under the perimeter ridge.

11. The apparatus of claim 2, the device has a rigid perimeter ring extends continuously around the perimeter of the perimeter ring.

12. The apparatus of claim 2, the rigid perimeter ring has the width, radius and diameter of the perimeter ridge.

13. The apparatus of claim 2, the rigid perimeter ring fits the circumference of the perimeter ridge.

14. The apparatus of claim 2, the rigid perimeter ring has an outwardly directed finger tab that will be held between fingers to attach and remove the rigid perimeter ring from the half-oval base below the perimeter line and perimeter ridge.

15. The apparatus of claim 2, the rigid plastic perimeter ring has an inwardly directed tongue tabs that will insert into matching tongue slots to permit the rigid plastic perimeter ring to attach to the half-oval base below the perimeter line against the perimeter ridge.

16. The apparatus of claim 2, the device has non-abrasive flexible material that will adjust, bend and flex;

17. The apparatus of claim 2, abrasives will be affixed on the outer surface of the flexible material;

18. The apparatus of claim 2, the flexible material with affixed abrasives will attach to the rigid perimeter ring that extends continuously around the perimeter of the perimeter ring;

19. The apparatus of claim 2, the flexible material with abrasives attached to the rigid perimeter ring that extends around the perimeter of the apparatus placed over the sponge rubber half-oval base attached to the perimeter ridge under the perimeter line will adjust, bend and flex equally and simultaneously when pressed on and moved over feet.

20. The apparatus of claim 3, the handheld portion has a flat bottom surface with hollow indentations projecting upward into the base of the handheld portion.

21. The apparatus of claim 3, the device has a separated half-oval base with a top flat surface.

22. The apparatus of claim 3, the device has a rigid plate with hollow rigid extensions that project upward from the top flat surface of the rigid plate.

23. The apparatus of claim 3, the device has a second rigid plate with a top flat surface with rigid extensions that project upward from the top flat surface.

24. The apparatus of claim 3, the second rigid plate has a bottom that will will attach to the top surface the half-oval device.

25. The apparatus of claim 3, the rigid extensions on the second rigid plate will detachably attach into the rigid extensions of the first rigid plate.

26. The apparatus of claim 4, the device is a rigid plastic bowl with upwardly curved top and bottom surfaces.

27. The apparatus of claim 4, the rigid plastic bowl has a spiral extension and a non-spiral extension connected to the top upwardly curved surface of the curved bowl.

28. The apparatus of claim 4, the bottom curved surface of the rigid plastic bowl has sponge rubber material attached to its bottom outer surface.

29. The apparatus of claim 4, the device with the bowl shape, with sponge rubber material on its outer surface has abrasives affixed on the outer surface of the sponge rubber.

30. The apparatus of claim 5, device has a spiral attachment that is attached to a rigid flat plate.

31. The apparatus of claim 6, the handheld device has elevated ridges to prevent movement and slippage of the fingers and hand.

32. The apparatus of claim 6, the device has a rigid plate attached to the flat bottom surface of the handheld device with elevated ridges.

33. The apparatus of claim 6, the device has individual pillows or rolls that extend downwardly from the bottom of the flat rigid plate.

34. The apparatus of claim 6, the individual abrasive pillows or rolls are equally separated and spaced apart.

35. The apparatus of claim 7, the device has double sponge rubber pillows.

36. The apparatus of claim 7, the device with double sponge rubber pillows will attach to bottom of the rigid plate.
